(19) United States
(12) Patent Application Publication
(10) Pub. No.: US 2023/0119206 A1
(43) Pub. Date: Apr. 20, 2023

(54) COMPOSITIONS COMPRISING IBRUTINIB AND AN ALKALOID HAVING ENHANCED BIOAVAILABILITY

(71) Applicant: CIPLA LIMITED, Mumbai (IN)

(72) Inventor: Geena Malhotra, Dadar (East) (IN)

(21) Appl. No.: 17/974,804

(22) Filed: Oct. 27, 2022

Related U.S. Application Data
(63) Continuation of application No. 16/648,727, filed on Mar. 19, 2020, now abandoned.

(30) Foreign Application Priority Data
Sep. 19, 2017 (IN) 201721033191

(51) Int. Cl. A61K 31/519; A61K 9/28; A61K 9/48; A61K 31/4525

(52) U.S. Cl. CPC A61K 31/519; A61K 9/28; A61K 9/4825; A61K 31/4525

(57) ABSTRACT

A pharmaceutical compositions provided for the treatment of proliferative disorders. The composition comprises a therapeutically effective amount of ibrutinib and a therapeutically effective amount of at least one alkaloid or derivative thereof. Methods and kits are also provided.

### CROSS REFERENCE TO RELATED APPLICATIONS

This application claims priority to co-pending Indian Provisional Patent Application Serial Number 201721033191 tiled on Sep. 19, 2017. This application is incorporated herein by reference, in its entirety.

### TECHNICAL FIELD OF INVENTION

The present invention relates to a pharmaceutical composition comprising ibrutinib or its pharmaceutically acceptable salts or its derivatives thereof and at least one pharmacokinetic booster or enhancer and optionally at least one pharmaceutically acceptable excipient. The present invention also provides manufacturing processes thereof and use of the said composition for prevention, treatment or prophylaxis of B-cell proliferative disorders in the patients in need thereof.

### BACKGROUND OF INVENTION

The drug absorption from a dosage form after administration depends on the release of the drug from the composition, the dissolution of the drug under physiological conditions, besides its permeability across the gastrointestinal tract. The formulation optimization approach for ibrutinib is mainly focused on enhancement of dissolution rate and solubility. A higher dissolution rate of a. composition generally increases release of the drug from its composition, which is a prerequisite for adequate bioavailability of a drug. Because of this requirement, a good in vitro dissolution of the composition may lead to good and adequate in vivo plasma concentration and therefore an adequate bioavailability.

Ibrutinib is novel BTK inhibitor currently approved and marketed under the brand name IMBRUVICA® 140 mg capsule for treatment of different kinds of B-cell proliferative disorders, in particular mantle-cell lymphoma (MCL) and chronic lymphocytic leukemia (CLL). The recommended dose of IMBRUVICA® is 560 mg (four 140 mg capsules) once daily for MCL and 420 mg (three 140 mg capsules) once daily for CLL. Thus, the required dose for MCL is comprised in four IMBRUVICA® capsules that must be administered orally once a day and the required dosage dose for CLL is comprised in three IMBRUVICA® capsules that have to be administered orally once a day. Bruton's tyrosine kinase (Btk), a member of the Tee family of non-receptor tyrosine kinases, is a key signaling enzyme expressed in all hematopoietic cells types except T lymphocytes and natural killer cells. Btk plays an essential role in the B-cell signaling pathway linking cell surface B-cell receptor (BCR) stimulation to downstream intracellular responses.

BTk is a key regulator of B-cell development, activation, signaling, and survival. In addition, BTk plays a role in a number of other hematopoietic cell signaling pathways, e.g., Toll like receptor (TLR) and cytokine receptor-mediated TNF-α production in macrophages, IgE receptor (FcεRI) signaling in Mast cells, inhibition of Fas/APO-1 apoptotic signaling in B-lineage lymphoid cells, and collagen-stimulated platelet aggregation

Ibrutinib (base) is practically insoluble in water and slightly soluble in HCl at pH 1.2. Ibrutinib is practically insoluble in non-polar solvents such as hexane and heptanes, sparingly soluble in ethyl acetate, ethanol and acetonitrile, soluble in acetone and methanol and freely soluble in N,N-dimethylformamide, tetrahydrofuran and dichloromethane. Thus, Ibrutinib can be classified as a BCS class II drug substance (low solubility-high permeability). Ibrutinib is represented by formula 1, as below;

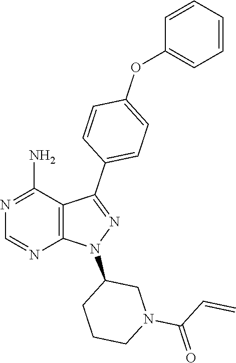 [SMILES: C=CC(=O)N1CCC[C@@H](n2nc(-c3ccc(Oc4ccccc4)cc3)c3c(N)ncnc32)C1]

Being a BCS class II drug, Ibrutinib is known to have bioavailability problems. The absolute bioavailability of ibrutinib is low at 2.9% with high inter-subject variability of the desired therapeutic response. The limited solubility of ibrutinib in aqueous media has been one challenge to obtain an orally bioavailable formulation.

In the past, some efforts are done to study the effect of different excipients acting to increase solubility and micronization of ibrutinib on rate of absorption in different formulations such as U.S. Pat. No. 9,545,407 discloses formulations of spray-dried ibrutinib dispersed in one or more solubility enhancers. The solubility enhancer can he a polymer matrix such as hydroxypropyl methyl cellulose acetate succinate (HPMCAS).

U.S. Pat. No. 9,296,753 relates to a crystalline form A of Ibrutinib having high aqueous solubility and pharmaceutical composition thereof with immediate release profile. The patent discloses Ibrutinib compositions containing 45.9 wt % of microcrystalline cellulose, 7.0 wt % of croscarmellose sodium, 4.2 wt % of sodium lauryl sulfate and 0.5 wt % of magnesium stearate. An approach of high dose was also suggested due to low bioavailability which is supposed to be responsible for the adverse side effects associated with the use of ibrutinib such as nausea or emesis, dizziness and diarrhea. The increase in oral bioavailability should enable administration of ibrutinib at a significantly lower therapeutically effective dose than is currently being used.

Hence, although some efforts have already been done to provide a highly bioavailable formulation of ibrutinib, there still exists a need to improve bioavailability of ibrutinib and a provide a formulation which maintaining the optimal concentration of ibrutinib and reducing the side effects exhibited by the same.

Pharmacokinetic boosters or enhancers are used to boost the effectiveness of drugs having low bioavailability. Without being bound to any theory, it is believed that when a pharmacokinetic booster or enhancer is coadministered with any such drug, the pharmacokinetic enhancer interferes with the breakdown of such drug, which causes the drug to remain in the body for a longer time and at a higher concentration.

Pharmacokinetic boosters or enhancers specifically cause inhibition of the enzyme system responsible for metabolism of drugs leading to an increase in the plasma concentrations of the co-administered drugs. These naturally occurring substances which act as bioenhancers are chemical entities that promote and augment the bioavailability of the drugs which are mixed with them and do not exhibit synergistic effect with the drug. Examples of these bioenhancers include piperine, garlic, Carum carvi, Currinum cyrrinurn lysergol, naringin, quercetin, niaziridin, glycyrrhizin, stevia, cow urine, distillate ginger, etc. These pharmacokinetic ‘boosters or ‘enhancers might reduce the cost of drug therapy, reduce pill burden for patients, and/or reduce the risk of sub therapeutic concentrations (e.g., development of resistance as well as enhance adherence to drug therapy). Further, use of a naturally occurring pharmacokinetic booster or enhancer would eliminate or reduce interactions with ibrutinib or other actives that would be concurrently administered and reduce the side effects caused by synthetic pharmacokinetic boosters.

There is no explicit teaching of improving the bioavailability of ibrutinib by incorporating piperine in the composition. Therefore, there remains a need to provide a new combination therapy of drug with low bioavailability such as ibrutinib or combination therapy of ibrutinib with pharmacokinetic booster or enhancer for the treatment of B-cell proliferative disorders which reduces the dose of ibrutinib and hence side effects exhibited by the same as well as maintaining the optimal concentration of the drag.

### OBJECT OF THE INVENTION

An object of the present invention is to provide a pharmaceutical composition comprising ibrutinib or its therapeutically acceptable salts or derivatives and piperine as a penetration or a bioavailability enhancer and optionally one or more pharmaceutically acceptable excipients.

Another object of the present invention is to provide a pharmaceutical composition comprising ibrutinib or its therapeutically acceptable salts or derivatives and piperine as a penetration or a bioavailability enhancer having improved solubility and therapeutic efficacy.

Yet another object of present invention is to provide a pharmaceutical composition composing ibrutinib or its therapeutically acceptable salts or derivatives and piperine as a penetration or a bioavailability enhancer for once or twice a day administration.

Another object of present invention is to provide a pharmaceutical composition comprising ibrutinib or its therapeutically acceptable salts or derivatives and piperine as a penetration or a bioavailability enhancer with reduced side effects and reduced dose.

Yet another object of the invention is to provide a process for manufacturing the pharmaceutical composition comprising ibrutinib or its therapeutically acceptable salts or derivatives and piperine as a penetration or a bioavailability enhancer optionally with one or more pharmaceutically acceptable excipients.

Another object of the present invention is to provide a composition comprising ibrutinib or its therapeutically acceptable salts or derivatives and piperine in the form of a kit.

Another object of the present invention is to provide a method for prevention and treatment or prophylaxis of B-cell proliferative disorders and process for manufacturing the pharmaceutical composition comprising composition comprising Ibrutinib or its therapeutically acceptable salts or derivatives and piperine as a penetration or a bioavailability enhancer optionally with one or more pharmaceutically acceptable excipients.

### SUMMARY OF THE INVENTION

An embodiment of the present invention is to provide a. pharmaceutical composition comprising ibrutinib or its therapeutically acceptable salts or derivatives and piperine as a penetration or a bioavailability enhancer and optionally one or more pharmaceutically acceptable excipients.

Another embodiment of the present invention is to provide a pharmaceutical composition comprising ibrutinib or its therapeutically acceptable salts or derivatives and piperine as a penetration or a bioavailability enhancer having improved therapeutic efficacy.

Yet another embodiment of present invention is to provide a pharmaceutical composition comprising ibrutinib or its therapeutically acceptable salts or derivatives and piperine as a penetration or a bioavailability enhancer for once or twice a day administration.

Another embodiment of present invention is to provide a pharmaceutical composition comprising ibrutinib and piperine as a penetration or a bioavailability enhancer with reduced side effects and reduced dose.

Yet another embodiment of the invention is to provide a process for manufacturing the pharmaceutical composition comprising ibrutinib or its therapeutically acceptable salts or derivatives and piperine as a penetration or a bioavailability enhancer optionally with one or more pharmaceutically acceptable excipients.

Another embodiment of the present invention is to provide a composition comprising ibrutinib or its therapeutically acceptable salts or derivatives and piperine in the form of a kit.

Another embodiment of the present invention is to provide a method for prevention and treatment or prophylaxis of B-cell proliferative disorders and process for manufacturing the pharmaceutical composition comprising composition comprising ibrutinib or its therapeutically acceptable salts or derivatives and piperine as a penetration or a bioavailability enhancer optionally with one or more pharmaceutically acceptable excipients.

### DETAILED DESCRIPTION OF INVENTION

For an effective therapeutic treatment, the drug should have high bioavailability with low dose leading to minimum side effects. Also, it is essential that the amount of drug reaches its site of action.

Bioavailability is defined as the rate and extent (amount) of absorption of unchanged drug from its dosage form. It is one of the important parameter to achieve desired concentration of drug in systemic circulation for pharmacological response to be shown.

A drug with poor bioavailability is one with poor aqueous solubility, slow dissolution rate in biological fluids, poor stability of dissolved drug at physiological pH, poor permeation through biomembrane, extensive presystemic metabolism. Poorly water soluble drugs often require high doses in order to reach therapeutic plasma concentrations after oral administration. Low aqueous solubility is the major problem encountered with formulation development of new chemical entities. Any drug to be absorbed must be present in the form of an aqueous solution at the site of absorption.

The drug like ibrutinib has very low bioavailability and thus there is a need for such formulation for better treatment of patients suffering from B-cell proliferative disorders.

Owing to the poor bioavailability of ibrutinib, efforts have been directed towards making a therapeutic effective formulation of ibrutinib having improved therapeutic effect. inventors of the present invention in their continuous effort of improving the bioavailability of ibrutinib or its pharmaceutically acceptable salts or its derivatives surprisingly found that improved bioavailability can he achieved by using natural penetration or bioavailability enhancer piperine with reduced dose and reduced side effects of ibrutinib.

The present invention provides a pharmaceutical composition comprising ibrutinib or its therapeutically acceptable salts or derivatives having increased therapeutic efficacy.

More particularly, the invention relates to a pharmaceutical composition comprising ibrutinib and a bioavailability enhancer or a penetration enhancer. The composition of the present invention is particularly useful for the treatment of B-cell proliferative disorders such as non-Hodgkin lymphoma (diffuse large B cell lymphoma, follicular lymphoma, mantle cell lymphoma or burkitt lymphoma), Waldenstrom macroglobulinemia, plasma cell myeloma, chronic lymphocytic leukemia, lymphoma, or leukemia. Ibrutinib compositions of the present invention may also be used for treatment of chronic graft versus host disease (cGVHD) after failure of one or more lines of systemic therapy.

Bioavailability enhancers, pharmacokinetic boosters, pharmacokinetic enhancers or penetration enhancers according to the present invention are drug facilitators, which by themselves do not show typical drug activity but when used in combination they enhance the activity of drug molecule in several ways including increasing permeability of the drug across the membrane, potentiating the drug molecule by conformational interaction, acting as receptors for drug molecule and making target cells more receptive to drugs. The term “pharmacokinetic booster or enhancer” may include an alkaloid. More particularly, the pharmacokinetic booster or enhancer” is piperine or its derivatives.

It is a known practice to use certain herbs either in combination or individually for enhancing the therapeutic effect of the active drug. There are many reports in which such drugs are combined with other drugs to increase the potency and therapeutic efficacy of the drug. There are decent number of studies that testify the use of herbs in the pharmaceutical field. Dutt et al, Materia Medica of Hindus, Calcutta (1900) mentions compositions containing herbs. Laksmipathi et al, One hundred useful drug, 3^{rd} Arogya Ashram Samithi, Madras (1946) has reported that the herbs are useful in correcting the balance of Kapha, Vata & Pitta, which according to experts of Ayurveda, are the three humors of the body, the imbalance of which, is responsible for causing diseases. Bose et al, Pharmacopia indica, Calcutta, 1928, discloses the property of long pepper for increasing efficacy of Vasaka as an anti-asthmatic agent.

Usha et al, Indian Drugs, 1982, (12), 476-479 has reported that Piper longum and Piper nigrum are almost equally effective as ginger in enhancing the bio-availability of the drug. U.S. Pat. No. 5,616,593 discloses compositions with increased bioavailability in which piperine is reported to be used in combination with antimicrobial agent, antiprotozoal agent, anthelmintic agent, central nervous system drug, non-steroid anti-inflammatory drug, antihistaminic, prokinetic drug, corticosteroid, steroid hormone, oral vaccine, haematinic, vitamin, antiulcer drug, muscle relaxant, or anticancer drug.

Without being bound by any theory whatsoever, the bio-enhancing property for piperine is due to its inherent attribute of making the target receptors more responsive to drugs, acting as receptors for drug molecules, increasing GIT vasculature by vasodilation to increase absorption of drugs, modulation of the cell membrane dynamics to increase transport of drugs across cell membranes etc. In some embodiments the present invention provides a low dose pharmaceutical composition comprising Ibrutinib which would ensure patient compliance due to simplification of therapy, ease of administration, an acceptable dosing regimen and bioavailability. The high bioavailability of the compositions of the present invention may be attributable to the bio-enhancing properties of Piperine or its derivatives such as tetrahydropiperine allowing faster and higher drug absorption. The dose which can be lower than the usual or the conventional dose, required to produce equal or higher therapeutic effect, may also reduce the side effects thereby leading to limit the risk to the patient.

The term “therapeutically effective amount” or “effective amount” is such that when administered, the pharmaceutical composition results in the inhibition of the systemic infection. The dosage administered to a patient can be as single or multiple doses depending upon a variety of factors, including the drug's administered pharmacokinetic properties, the route of administration, patient conditions and characteristics (sex, age, body weight, health, size, etc.), and extent of symptoms, concurrent treatments, frequency of treatment and the effect desired.

The term “treatment” or “treating” of a disease, or condition refers to executing a protocol that may include administering one or more drugs to a patient, in an effort to alleviate signs or symptoms of the disease or condition. Alleviation can occur prior to signs or symptoms of the disease or condition appearing, as well as after their appearance. Thus, treating or treatment includes reducing, preventing or prevention of the disease or condition. In addition, treating or treatment does not require complete alleviation of signs or symptoms, does not require a cure, and specifically includes protocols that have only a marginal effect on the patient.

The term “Ibrutinib” is used in broad sense to include not only “Ibrutinib” per se but also its pharmaceutically acceptable derivatives thereof. Suitable pharmaceutically acceptable derivatives include pharmaceutically acceptable salts, solvates, hydrates, anhydrates, enantiomers, esters, isomers, polymorphs, prodrugs, tautomers, complexes etc.

Ibrutinib may be administered at least once, twice or thrice a day in the dosing range from about 10 mg to about 1200 mg. Preferably, Ibrutinib may be administered at least once, twice or thrice a day in the dosing range from about 10 mg to about 800 mg. Preferably, Ibrutinib may be administered at least once, twice or thrice a day in the dosing range from about 10 mg to about 600 mg. Preferably Ibrutinib may be administered at least once, twice or thrice a day in the dosing range from about 10 mg to about 560 mg. Preferably Ibrutinib may be administered at least once, twice or thrice a day in the dosing range from about 10 mg to about 420 mg. Preferably ibrutinib may be administered at least once, twice or thrice a day in the dosing range from about 10 mg to about 280 mg. Preferably Ibrutinib may be administered at least once, twice or thrice a day in the dosing range from about 10 mg to about 200 mg. Preferably ibrutinib may be administered at least once, twice or thrice a day in the dosing range from about 10 mg to about 140 mg. Preferably ibrutinib may be administered at least once, twice or thrice a day in the dosing range from about 10 mg to about 1000 mg. The preferred dosing range for Ibrutinib may be from about 25 mg to about 750 mg once daily.

In more preferred embodiments, the dose of Ibrutinib may be from about mg/kg/day to about 13 mg/kg/day. In more preferred embodiments, the dose of Ibrutinib may be from about 2.5 mg/kg/day to about 10 mg/kg/day. In more preferred embodiments, the dose of Ibrutinib may be from 2.5 mg/kg/day to about 6 mg/kg/day, In some embodiments, the dose of Ibrutinib may be from about 2.5 mg/kg/day to about 4 mg/kg/day. In even more preferred embodiment, the dose of Ibrutinib is about 2.5 mg/kg/day.

The fruit of black pepper (Piper nigrum L.) and long pepper (Piper longum L.) are both important medicinal herbs in Ayurvedic and Unani (traditional Indian) systems of medicine, wherein the remedy generally consists of mixtures of herbs. A wide range of the medicinal uses of black pepper are known and have been documented including its use in the treatment of leucoderma.

Piperine, the major alkaloid found in the fruit of black pepper (Piper nigrum L.; Piperaceae), stimulates the replication of melanocytes and induces the formation of melanocytic; dendrites. Piperine is expected to cause the repopulation of vitiligo patches through a stimulatory effect on perilesional and follicular melanocytes.

In some embodiments, the pharmacokinetic booster or enhancer comprises piperine, tetrahydropiperine, cis-piperine, trans-piperine, cis-trans piperine, trans,cis-piperine, cis,cis-piperine, trans,trans-piperine or a combination thereof. More preferably, the pharmacokinetic booster or enhancer is piperine or tetrahydropiperine and its analogs or derivatives.

Piperine, (E.E.) 1˜[5,3-benzodioxyl-5-yl)-1-oxo-2,4-pentadienyl-piperidine, of the formula I is a naturally occurring alkaloid and is the main constituent of many Piper species. It is mostly obtained from Piper longum (3-5%) or Piper nigrum (3-9%) which are cultivated on a large scale in India and therefore readily available

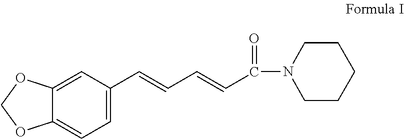 [SMILES: O=C(/C=C/C=C/c1ccc2c(c1)OCO2)N1CCCCC1]

Piperine inhibits both p-glycoprotein, major drug metabolizing enzyme CYP3A4 and phase II reactions in animal models. Its chemico-biological interactions enhances the bioavailability of various structurally and therapeutically different drugs.

Piperine has also been reported to occur in other Piper species i.e. P. Acutisleginum, album, argyrophylum, attenatum, aurantiacum, betle, callosum, chaba, cubeba, guineense, hancei, khasiano, longum, macropodum, nepalense, novae hollandiae, peepuloides, retrokacturn, sylvalictum.

Piperine is reported to be a bioavailability enhancer and thus may be explored to improve the pharmacokinetic activity of certain drugs which has inherent problems with permeability. Role of Piperine As A Bioavailability Enhancer, UMESH K PATIL et al International Journal of Recent Advances in Pharmaceutical Research October 2011; 4: 16-23 discloses piperine as a bioavailability enhancer.

Tetrahydropiperine is a structural analog of Piperine. The two double bonds at position 2 and 4 are saturated to give a tetrahydro analog. Tetrahydropiperine is chemically known as 5-(1,3-benzodioxol-5-yl)-1-piperidin-1-ylpentan-1-one and is structurally represented as shown below.

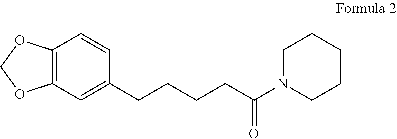 [SMILES: O=C(CCCCc1ccc2c(c1)OCO2)N1CCCCC1]

Tetrahydropiperine occurs like piperine naturally in black pepper (about 0.7% in black pepper oleoresin). Tetrahydropiperine can he synthesized from piperine which is previously extracted from black pepper oleoresin. The term “analogs or derivatives” of tetrahydropiperine is used in broad sense to include alkyltetrahydropiperines, e.g. methyltetrahydropiperine or ethyltetrahydropiperine, dialkyltetrahydropiperines, e.g. dimethyltetrahydropiperine or diethyltetrahydropiperine, alkoxylated tetrahydropiperine, e.g. methoxy tetrahydropiperine, hydroxylated tetrahydropiperine, e.g. 1-[(5,3-benzodioxyl-5-yl)-1-hydroxy-2,4-pentadienyl]-piperine, 1-[(5,3-benzodioxyl-5-yl)-1-methoxy-2,4-pentadienyl]-piperine, halogenated tetrahydropiperine, e.g. 1-[(5,3-benzodioxyl-5-yl)-1-oxo-4-halo-2-pentenyl]-piperine and 1-[(5,3-benzodioxy-5-yl)-1-oxo-2-halo-4-pentertyl]-piperine, dihydropiperine, alkyldihydropiperines, e.g. methyldihydropiperine or ethyldihydropiperine, dialkyldihydropiperines, e.g. dimethyldihydropiperine or diethyldihydropiperine, alkoxylated dihydropiperine, e.g. methoxy dihydropiperine, and halogenated dihydropiperine and their pharmaceutically acceptable salts, pharmaceutically acceptable solvates, pharmaceutically acceptable hydrates, pharmaceutically acceptable anhydrates, pharmaceutically acceptable enantiomers, pharmaceutically acceptable esters, pharmaceutically acceptable isomers, pharmaceutically acceptable polymorphs, pharmaceutically acceptable prodrugs, pharmaceutically acceptable tautomers, pharmaceutically acceptable complexes etc. In some embodiments, preferably the dose of piperine ranges from about 0.5 mg to about 400 mg and the dose of tetrahydropiperine ranges from about 0.5 mg to about 400 mg. In some embodiments, the dose of the piperine and/or the tetrahydropiperine ranges from about 0.5 mg, 1, 2, 3, 4, 5, 7, 8, 9, 10, 15, 20, 25, 30, 35. 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, to about 400 mg.

Preferably the dose of ibrutinib ranges from about 10 mg to about 250 mg and that of piperine ranges from about 5 mg to about 100 mg.

In some embodiments, the ratio of Ibrutinib to the at least one pharmacokinetic booster or enhancer such as piperine is from about 100:1 to about 1:1 by weight.

Piperine is expected to improve the absorption of ibrutinib multifold in the inventive composition and thus lead to increase in overall bioavailability. Preferably, piperine improves the absorption 1-20 times, more preferably, 2-10 times. In a preferred embodiment, the piperine improves the absorption of Ibrutinib around 4 to 5 times.

Piperine does not appear to have a role in treating any disease and apparently does not exhibit anticancer medicinal properties. It is therefore surprising that it causes a synergistic effect in increasing the bioavailability of the Ibrutinib and thereby renders it more therapeutically effective. It would be observed from the above description that piperine when mixed with Ibrutinib produces synergistic effects resulting in a composition which has enhanced bioavailability of the drug and consequently helps in reducing the quantity of drug to be administered to the patient for producing the same therapeutic effect. Such an effect will avoid unnecessary administration of the drug to the patient, which will help in minimizing, reducing or eliminating the adverse effect the drug might have on the patient. In other words, such a combination increases the therapeutic index of ibrutinib. Therefore, the combination of piperine and Ibrutinib is not a mere admixture of the ingredients employed in the process resulting in the mere aggregation of the properties of the ingredients.

In some embodiments, the at least one pharmacokinetic booster or enhancer or derivative thereof increases the bioavailability of ibrutinib or its pharmaceutically acceptable salts or derivatives thereof from about 10% to about 100%, from about 10% to about 70%, from about 10% to about 50%, from about 10% to about 30%, or from about 10% to about 20%. In some embodiments, the at least one pharmacokinetic booster or enhancer or derivative thereof increases the bioavailability of ibrutinib or its pharmaceutically acceptable salts or derivatives thereof from about 10%, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%. In some embodiments, the at least one pharmacokinetic booster or enhancer or derivative thereof decreases the dose of ibrutinib or its pharmaceutically acceptable salts or derivatives thereof from about 5% to about 95%, from about 10% to about 90%, from about 20% to about 80%, from about 30% to about 70%, from about 40% to about 60%, or to about 50% and still providing the desired therapeutic effect.

In an embodiment, the compositions of the present invention comprising Ibrutinib or its pharmaceutically acceptable salts or its derivatives thereof and piperine or its pharmaceutically acceptable derivatives show improved bioavailability and hence improved therapeutic efficacy compared to the marketed formulation of Ibrutinib.

Preferably, the pharmaceutical compositions comprising ibrutininb and piperine may be administered once or twice a day.

Preferably, the pharmaceutical compositions comprising ibrutinib and piperine may be administered once or twice a day with reduced dose.

In another embodiment, the present invention also provides a process for the preparation of pharmaceutical compositions comprising Ibrutinib or its pharmaceutically acceptable salts or derivatives thereof and piperine or its pharmaceutically acceptable thereof having increased therapeutic efficacy.

The pharmaceutical preparations are prepared by mixing a drug along with piperine. In a preferred embodiment of the invention, the quantity of piperine used may vary from 0.1% to 50% by weight of the drug. More preferably the amount of piperine may vary from 0.1% to 20% by weight of the drug. The amount of the drug in the composition may vary from 10% to 95% by weight of the composition. The remaining 90% to 5% of the composition is made up of piperine and as necessary pharmaceutically acceptable inert excipients, vehicles diluents and/or binding agents.

The present invention also provides for the administration of additional therapeutic agent with ibrutinib to be administered simultaneously or separately either in the same or different pharmaceutical compositions. If there is separate administration, the invention furthermore provides that the subsequently administered therapeutic agents should be administered to a patient within a time scale to achieve, or more particularly optimize, synergistic therapeutic effect of such a combined preparation.

The inventors of the present invention have also found that the bioavailability properties of the ibrutinib may also be improved by nanosizing. In some embodiments, the pharmaceutical composition is administered via nanoparticles having a size of about 1 nanometer (nm) to about 50 nm. In some embodiments, the nanoparticles have a size of from about 1, 2, 3, 4, 5, 6, 7, 8, 9, 10, 11, 12, 13 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nm.

The term “pharmaceutical composition” includes dosage forms such as but not limited to, unit dosage forms including tablets, capsules (filled with powders, pellets, beads, mini-tablets, pills, micro-pellets, small tablet units, multiple unit pellet systems (MUPS), disintegrating tablets, dispersible tablets, granules, and microspheres, multiparticulates), sachets (filled with powders, pellets, beads, mini-tablets, pills, micro-pellets, small tablet units, MUPS, disintegrating tablets, dispersible tablets, granules, and microspheres, multiparticulates), powders for reconstitution, transdermal patches and sprinkles, however, other dosage forms such as controlled release formulations, lyophilized formulations, modified release formulations, delayed release formulations, extended release formulations, pulsatile release formulations, dual release formulations and the like. Liquid or semisolid dosage form (liquids, suspensions, solutions, dispersions, ointments, creams, emulsions, microemulsions, sprays, patches, spot-on), injection preparations, parenteral, topical, inhalations, buccal, nasal etc. may also be envisaged under the ambit of the invention.

Preferably, the mini-tablets or granules filled in such hard gelatin capsules or sachets are directly administered or by sprinkling the mini-tablet or granules on regular meals. Alternatively, the mini-tablets or granules filled in hard gelatin capsules or sachets may be administered with liquid or semi-solid beverages such as but not limited to, juices, water.

The mini-tablets or granules, according to the present invention, may also optionally be coated. Preferably, mini-tablets or granules, according to the present invention, may be film coated. More preferably, the mini-tablets or granules may be seal coated and then film coated and further filled in hard gelatin capsules or sachets.

It is further well known in the art that a tablet formulation is the preferred solid dosage form due to its greater stability, less risk of chemical interaction between different medicaments, smaller bulk, accurate dosage, and ease of production.

Tablet forms may include one or more of lactose, sucrose, mannitol, corn starch, potato starch, alginic acid, microcrystalline cellulose, acacia, gelatin, guar gum, colloidal silicon dioxide, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms may comprise the composition in a flavor, usually sucrose and acacia or tragacanth, as well as pastilles comprising the composition in an inert base, such as gelatin and glycerin, or sucrose and acacia, emulsions, gels, and the like.

Solid unit dosage forms, according to the present invention, are preferably in the form of tablets either single or bilayered or multilayered tablets but other conventional dosages such as powders, pellets, capsules and sachets may fall within the scope of this invention. According to another embodiment, the pharmaceutical composition may be administered as a single layered or bilayererd or multilayered tablet wherein each layer may or may not contain ibrutinib along with pharmaceutically acceptable excipients which are then compressed to provide either a single layered, bilayered or multilayered tablet.

According to one embodiment, the composition of present invention comprising ibrutinib or its pharmaceutically acceptable salts or derivatives thereof with other therapeutically active drugs may be administered simultaneously, separately or sequentially in a single unit dosage form. When the active ingredients are administered sequentially, either at least ibrutinib or piperine/tetrahydropiperine, may be administered first. When administration is simultaneous, the active ingredients may he administered either in the same or different pharmaceutical compositions. Adjunctive therapy, i.e. where one active ingredient is used as the primary treatment and the other active ingredient(s) is/are used to assist that primary treatment is also an embodiment of the present invention.

Accordingly, when the pharmaceutical composition is provided in unit dosage forms, as discussed above, the unit dosage form can be uncoated or coated.

Liquid formulations may include diluents, such as alcohols, for example, ethanol, benzyl alcohol, and the polyethylene alcohols, either with or without the addition of a pharmaceutically acceptable surfactant, suspending agent, or emulsifying agent.

Capsule forms may be of the ordinary hard or soft shelled gelatin type containing, for example, surfactants, lubricants, and inert fillers, such as lactose, sucrose, calcium phosphate, and corn starch. Preferably, the dosage form of the present invention is in the form of soft gelatin capsule.

Suitable excipients may be used for formulating the dosage forms according to the present invention such as, but not limited to, surface stabilizers or surfactants, viscosity modifying agents, polymers including extended release polymers, stabilizers, disintegrants or super disintegrants, diluents, plasticizers, binders, glidants, lubricants, sweeteners, flavoring agents, anti-caking agents, opacifiers, anti-microbial agents, antifoaming agents, emulsifiers, buffering agents, coloring agents, carriers, fillers, anti-adherents, solvents, taste-masking agents, preservatives, antioxidants, texture enhancers, channeling agents, coating agents or combinations thereof. In one of the preferred embodiment, pharmaceutically acceptable excipients like carriers, thickening agents, surfactants can be used in the preparation of soft gelatin capsules containing Ibrutinib and piperine or tetrahydro piperine. It is preferred that the pharmaceutically acceptable carrier be one which is chemically inert to the piperine and ibrutinib and one which has no detrimental side effects or toxicity under the conditions of use. Suitable carriers include but not limited to corn oil glycerides, ethyl oleate, glycerol mono/dioleate, glycerol monolinolate, Glycerol monocaprylocaprate, macrogolglycerol caprylocaprate, macrogolglycerol linoleate, Tocopheryl polyethylene glycol 1000 succinate (Vitamin E TPGS), Caprylocaproyl macrogol-8 glycerides, medium chain partial glycerides, medium chain triglycerides, caprylic-capric triglycerides, caprylic/capric/inoleic triglycerides, caprylic/capric/succinic triglycerides, Glycerol Tricaprylate/Caprate (Captex 355), propylene glycol dicaprylateidicaprate, oleic acid polyoxyl castor oil, polyoxyl hydrogenated castor oil, propylene glycol, propylene glycol monolaurate, Propylene glycol monocaprylate (Gelucire 33/01), refined animal derived oil, Glycerol monolinoleate (winterized oil), refined soybean oil, refined vegetable oil, sorbitan monostearate, triacetin, triethyl citrate, or mixtures thereof. Medium chain triglycerides like Miglyol 810, 812 are preferred. Suitable thickeners include but not limited to semisolid highly viscous or solid polyethyleneglycols (e.g. polyethylene 1000 to 20000), preferably polyethyleneglycols 1000 to 6000, preferably polyethyleneglycol 4000, or oleogel forming excipients, such as Colloidal Silica or Bentonite, or lipophilic or amphiphilic excipients of high viscosity, such as bees wax, glycerol monostearate, semisolid lanolin, hydrogenated vegetable oil, partially hydrogenated vegetable oil or hard fats, hard fats with hydroxyl values between 20-50. Hard fat as thickener is preferred. Suitable surfactants include but not limited to surfactants such as nonionic, ionic surfactants including Glycerol monooleates, polyoxyethylene sorbitan esters, polyoxyethylene alkyl ethers, Highly purified diethyl ene glycol monoethyl ether, polyoxyethylene stearates, 1, 2 Propanediol Monocaprylate, poloxamers, Caprylocaproyl macrogol-8 glycerides, Polyethylene glycol sorbitan monooleate (tween 80), lecithin and the like. Soya lecithin is the preferred surfactant.

In one of the preferred embodiment, the pharmaceutical composition comprises of a soft gelatin capsule, in which ibrutinib is suspended in a vehicle comprising of medium chain triglycerides and piperine or derivative thereof (tetrahydropiperine).

In another preferred embodiment, the pharmaceutical composition comprises of a soft gelatin capsule, in which ibrutinib is suspended in a vehicle comprising of medium chain triglycerides, piperine, a surfactant and a thickening agent.

The present invention provides method of prevention, treatment or prophylaxis of B-cell proliferative disorders which method comprises administering the pharmaceutical composition substantially as hereinbefore described.

There is further provided by the present invention a pharmaceutical composition comprising ibrutinib and piperine as hereinbefore described, for use in treating disorders or conditions that respond to, or are prevented, ameliorated or eliminated by administering the pharmaceutical composition comprising substantially as hereinbefore described

Though the efficacy of the composition has more effect when piperine and the drug are administered in one single composition, the possibility of administering the required quantity of the drug and piperine separately is also envisaged according to this invention. In other words, the drug and piperine may be administered to the patient separately. However, it is preferred to use the composition as a single dosage form. It is also preferred that the composition be administered orally. If the drug and piperine are administered separately, it is also preferred that they be administered orally.

The nature of the invention, its objects and advantages are explained hereunder in greater detail in relation to non-limiting exemplary embodiments.

The following examples are for the purpose of illustration of the invention only and are not intended in any way to limit the scope of the present invention.

### EXAMPLE 1

### Ibrutinib-Piperine Soft Gel Capsule

| Ingredients | Quantity in mg |
|---|---|
| Ibrutinib | 20-140 |
| Soya lecithin | 0.1-5.0 |
| Medium chain triglyceride | 10-90 |
| (Miglyol 810[triglycerides of the fractionated plant fatty | |
| acids C8 and C10], Captex 355 (Glycerol | |
| Tricaprylate/Caprate) | |
| Hard fat -- Gelucire 33/01[Propylene glycol | 20-80 |
| monocaprylate], Witepsol W 35 9 [Hard fats], | |
| Softisan 378 [Semisolid lanolin] | |
| Piperine or Tetrahydro Piperine | 0.1-70 |

Procedure:
1. Soya lecithin, Medium chain triglyceride, hard fat and Piperine or Tetrahydro Piperine were dispensed in a container and mixed using a suitable stirrer.
2. Ibrutinib was added to the above mixture under continuous stirring till a clear homogenous solution was obtained.
3. The liquid formed in the above step was encapsulated in a soft gel capsule of suitable size.

### EXAMPLE 2

### Ibrutinib-Piperine Soft Gel Capsule

| Ingredients | Quantity in mg |
|---|---|
| Ibrutinib | 20-140 |
| Maisine (Glycerol monolinoleate) | 10-300 |
| Labrasol (Caprylocaproyl macrogol-8 glycerides ) | 10-300 |
| Tween 80 (Polyethylene glycol sorbitan monooleate) | 10-300 |
| Vit E TPGS (Tocopheryl polyethylene glycol 1000 | 0.2-10 |
| succinate) | |
| Piperine or Piperin tetrahydrate | 0.1-70 |
| Total | 1000 |

Procedure:
1. Maisine, Labrasol, Tween 80, Vit E TPGS and Piperine der Piperine tetrahydrate were dispensed in a suitable container and subsequently mixed using a suitable stirrer.
2. Ibrutinib was added to the above mixture under continuous stirring condition until a clear homogenous solution was obtained.
3. The liquid formed in the above process step was encapsulated in a soft gel capsule of suitable size.

### EXAMPLE 3

### Ibrutinib-Piperine Soft Gel Capsule

| Ingredients | Quantity in mg |
|---|---|
| Ibrutinib | 20-140 |
| Capmul (Glycerol monocaprylocaprate) | 100-500 |
| Peceol (Glycerol monooleates) | 10-500 |
| Capryol 90 (1,2 Propanediol Monocaprylate) | 50-700 |
| Labrasol (Caprylocaproyl macrogol-8 glycerides) | 50-700 |
| Propylene Glycol | 10-200 |
| Trancutol HP (Highly purified diethylene glycol | 10-200 |
| monoethyl ether) | |
| Piperine or Piperin tetrahydrate | 0.1-70 |
| Shell Part | % w/w |
| Gelatin | 20-70 |
| Glycerol | 5-50 |
| Titanium Dioxide | 0.05-1.0 |

Procedure:
1. Capmul, Peceol, Capryol 90, Labrasol, Propylene Glycol, Trancutol HP were dispensed in a container arid mixed using suitable stirrer.
2. Ibrutinib was added to the above mixture under continuous stirring conditions until a clear and homogenous solution was obtained.
3. The liquid formed in the above process step was encapsulated in a soft gel capsule of suitable size.

### EXAMPLE 4

### Ibrutinib-Pipeline Tablets

| Sr. No. | Ingredients | Quantity mg/tablet |
|---|---|---|
| 1 | Ibrutinib | 25-750 mg |
| 2 | Piperine | 5-75 |
| 3 | Glycerol palmitostearate | 10-150 |
| 4 | Microcrystalline cellulose (Avicel PH | 50-200 |
| | 5-100 101) | |
| 5 | Silicon dioxide colloidal (Aerosil 200) | 40-170 |
| 6 | Edetate disodium | 1-7.5 |
| 7 | Sodium starch Glycolate | 30-60 |
| 8 | Magnesium stearate | 3-10 |
| 9 | Talc | 2-5 |

Coating

| | | |
|---|---|---|
| 10 | Opadry ready mix | 10-45 |
| 11 | Purified water | qs |

Procedure:
Ibrutinib, Piperine, microcrystalline cellulose, colloidal silicon dioxide, sodium starch glycolate, edetate disodium were sifted and added to a suitable blender. Edetate disodium, Glycerol palmitostearate, Magnesium stearate and talc were sifted and added to the blend obtained above, mixed, compressed into tablets and coated.

It will he readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be failing within the scope of the invention.

It is to be understood that the phraseology and terminology used herein is for the purpose of description and should not he regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise.

1. A pharmaceutical composition comprising a therapeutically effective amount of ibrutinib, a therapeutically effective amount of at least one alkaloid or derivative thereof and optionally one or more therapeutically acceptable excipient.

2. The pharmaceutical composition of claim 1, wherein the at least one alkaloid comprises piperine, tetrahydropiperine, cis-piperine, trans piperine, cis-trans piperine, trans,cis-piperine, cis,cis-piperine, trans, trans piperine or a combination thereof.

3. The pharmaceutical composition of claim 1, wherein the amount of ibrutinib in the composition is from about 10 mg to about 1000 mg.

4. The pharmaceutical composition of claim 2, wherein the piperine is in the composition from about 0.5 mg to about 400 mg.

5. The pharmaceutical composition of claim 1, wherein the ratio of ibrutinib and piperine is from about 100:1 to about 1:1 by weight.

6. The pharmaceutical composition of claim 1, wherein the composition is in the form of a tablet, mini-tablet, granules, sprinkles, capsules, sachets, powders, pellets, disintegrating tablets, dispersible tablets, solution, suspension, emulsion, lyophilized powder or in the form of a kit.

7. The composition of claim 1, wherein administration of the composition to a patient in need thereof produces a greater plasma concentration of ibrutinib compared to an otherwise same composition not including an alkaloid.

8. The pharmaceutical composition of claim 1, wherein the bioavailability of ibrutinib is increased from about from about 10% to about 100%.

9. The method of claim 11, wherein the composition comprises less than 140 mg of ibrutinib.

10. The pharmaceutical composition method of claim 9, wherein the dose of ibrutinib is decreased from about from about 5% to about 95% of 140 mg.

11. A method of treating B-cell proliferative disorders in a patient in need of such treatment, the method comprising: administering a pharmaceutical composition comprising (i) a therapeutically effective amount of ibrutinib; (ii) a therapeutically effective amount of at least one alkaloid or derivativethereof; and (iii) one or more pharmaceutically acceptable excipients comprising earners, diluents, fillers, binders, lubricants, glidants, disintegrants, bulking agents, flavorants or any combination thereof.

12. The method according to claim 11, wherein the B-cell proliferative disorders are non-Hodgkin lymphoma (diffuse large B cell lymphoma, follicular lymphoma, mantle cell lymphoma or burkitt lymphoma), Waldenstrom macroglobulinemia, plasma cell myeloma, chronic lymphocytic leukemia, lymphoma, or leukemia.

13. The method according to claim 11, wherein the at least one alkaloid comprises piperine, tetrahydropiperine, cis-piperine, trans-piperine, cis trans piperine, trans,cis-piperine, cis,cis-piperine, trans,trans-piperine or a combination thereof.

14. A kit for treating B-cell proliferative disorders, the kit comprising a therapeutically effective amount of ibrutinib and a therapeutically effective amount of at least one alkaloid or derivative thereof, wherein the ibrutinib is in a separate composition from the at least one alkaloid or derivative thereof.

15. The method of claim 14, wherein (i) the ibrutinib is in a first composition and the at least one alkaloid or derivative thereof is in a second composition; or (ii) ibrutinib and the at least one alkaloid or derivative thereof is combined in one composition.
